(19) United States
(12) Patent Application Publication
(10) Pub. No.: US 2026/0159669 A1
(43) Pub. Date: Jun. 11, 2026

(54) AMINOLYSIS OF POLYESTERS AND POLYETHYLENE TEREPHTHALATE-DERIVED ASPHALT MODIFIERS

(71) Applicants: Arizona Board of Regents on behalf of Arizona State University, Scottsdale, AZ (US); Alliance for Sustainable Energy, LLC, Golden, CO (US)

(72) Inventors: Husain Mithaiwala, Tempe, AZ (US); Adarsh Bakkireddy, Hillsboro, OR (US); Matthew Green, Phoenix, AZ (US); Robert David Allen, Golden, CO (US)

(21) Appl. No.: 19/182,095

(22) Filed: Apr. 17, 2025

Related U.S. Application Data
(60) Provisional application No. 63/635,306, filed on Apr. 17, 2024.
(60) Provisional application No. 63/707,360, filed on Oct. 15, 2024.

(51) Int. Cl. C08K 5/20; C07C 231/02; C08L 95/00

(52) U.S. Cl. CPC C08K 5/20; C07C 231/02; C08L 95/00; C08L 2555/60

(57) ABSTRACT

Depolymerizing a polyester material to yield a terephthalamide includes combining the polyester material, an alkylamine, and a mediator to yield a reaction mixture, heating the reaction mixture, and maintaining a reaction for a reaction time to yield the terephthalamide. In one example, the mediator is ethylene glycol. Preparing a modified asphalt binder includes combining the terephthalamide with an asphalt binder to yield a modified asphalt binder precursor and heating the modified asphalt binder precursor to yield a modified asphalt binder.

### CROSS-REFERENCE TO RELATED APPLICATIONS

This application claims the benefit of U.S. Patent Application No. 63/635,306 filed on Apr. 17, 2024, and 63/707,360 filed on Oct. 15, 2024, each of which is incorporated by reference herein in its entirety.

### STATEMENT OF GOVERNMENT SUPPORT

This invention was made with government support under DE-AC36-08GO28308 awarded by the Department of Energy. The government has certain rights in the invention.

### TECHNICAL FIELD

This invention relates to aminolysis of polyesters and polyethylene terephthalate (PET)-derived modifiers for asphalt binders.

### BACKGROUND

Polyethylene terephthalate (PET) plastics are some of the most utilized plastics in the world. PET is a common component in disposable water bottles and is also a common component in textile manufacturing industries. These products usually end up in landfills and are seldom recycled.

### SUMMARY

This disclosure describes aminolysis of polyesters such as polyethylene terephthalate (PET) and PET copolymers with ethylene glycol and primary long-chain alkylamines (e.g., C4-C18) and branched alkylamines (e.g., isobutylamine, isopentylamine, (2-methylbutyl)amine, and 2-ethyl-1-hexylamine) to yield corresponding mono- and di-amides of terephthalic acids (e.g., terephthalamides). The reaction is shown to go to 100% completion in the presence of ethylene glycol at low reaction temperatures (e.g., 125° C.) and short reaction times (e.g., 6 hours). Monomers suitable for use with PET copolymers include cyclohexanedimethanol. The products of the aminolysis process of PET using long-chain amines can increase the fatigue and rutting factor of asphalt. The products can also be used to increase the strength in concrete binders and as intermediary products in the manufacturing of unsaturated polyester polyurethane and unsaturated polyester resins. The generated terephthalamides can be utilized as phase change materials in several industries.

This disclosure also describes the use of two terephthalamide asphalt modifiers-N¹,N⁴-bis(2-hydroxyethyl)terephthalamide (HETP) and N¹,N⁴-(dioctyl)terephthalamide (OTP) derived from end-of-life plastics, on the properties of asphalt binders. Both modifiers, synthesized from waste PET, exhibit distinct molecular characteristics that affect their performance as asphalt additives. Multiple stress creep recovery tests reveal contrasting effects of HETP and OTP on the rheological behavior of the asphalt binder in terms of strain accumulation under repeated loading. These measurements provide an insight into the asphalt binder's ability to recover from deformation due at least in part to continuous vehicular traffic.

The OTP-modified binder exhibits reduced strain accumulation compared to the unmodified binder, suggesting improved elastic recovery. In contrast, the HETP-modified binder becomes less elastic, leading to increased strain accumulation, highlighting that not all plastic-derived modifiers contribute positively to asphalt performance. Density functional theory (DFT) calculations provide insights into these differences. OTP, with its polar amide groups and non-polar eight-carbon arms, disperses among asphaltene and resin molecules, enhancing intermolecular interactions. The interaction energy of OTP with asphaltene within the asphaltene-OTP-resin ternary system (−52.1 kcal/mol) closely matches the asphaltene-asphaltene interaction energy (−52.5 kcal/mol), resulting in stable complexes that can prevent further asphaltene aggregation. In contrast, HETP's interactions with asphaltenes suggest that the HETP modification is insufficient to disrupt existing asphaltene aggregation. HETP can bind to the binder's polar components but lacks adequate dispersion, creating a less homogeneous and more deformation-prone binder as supported in the strain accumulation measurements after repeated loading. This disclosure provides an insight into the role of molecular characteristics in determining the effectiveness of plastic-derived modifiers in asphalt binders. The results suggest that while PET-derived modifiers can be engineered to improve asphalt durability and longevity, not all plastic-derived modifiers improve asphalt durability.

In a first general aspect, depolymerizing polyester material to yield a terephthalamide includes combining the polyester material, an alkylamine, and a mediator to yield a reaction mixture, heating the reaction mixture, and maintaining a reaction for a reaction time to yield the terephthalamide. The mediator is ethylene glycol.

Implementations of the first general aspect can include one or more of the following features. The polyester material can include polyethylene terephthalate (PET), polyethylene terephthalate-glycol, poly(trimethylene terephthalate), or any combination thereof. In some cases, the alkylamine includes butylamine, amylamine, hexylamine, heptylamine, octylamine, nonylamine, decylamine, undecylamine, dodecylamine, tridecylamine, tetradecylamine, pentadecylamine, hexadecylamine, heptadecylamine, octadecylamine, isobutylamine, isopentylamine, (2-methylbutyl)amine, 2-ethyl-1-hexylamine, or any combination thereof. In certain cases, the terephthalamide includes N¹, N⁴-dioctylterephthalamide (OTP), N¹, N⁴-didodecylterephthalamide (DTP), N¹, N⁴-dihexadecylterephthalamide (HTP), or any combination thereof. A weight ratio of polyester:alkylamine:mediator in the reaction mixture can be in a range of about 1:1:4 to 1:4:4. In certain implementations, a molar ratio of polyester:alkylamine in the reaction mixture is in a range of 1:2 to 1:10. In some cases, a molar ratio of polyester:mediator is in a range of 1:2 to 1:24. The heating can include heating to a temperature in a range of 100° C. to 200° C. or heating to a temperature in a range of 120° C. to 130° C. The reaction time can be between 1 hour and 72 hours or between 1 hour and 10 hours. In some cases, the reaction mixture is contained in a reaction vessel, and further includes purging the reaction vessel with an inert gas. The inert gas can include nitrogen.

The first general aspect can further include isolating the terephthalamide. In some cases, the isolating the terephthalamide includes combining the terephthalamide with a washing agent. The washing agent can include acetone. In certain implementations, the isolating the terephthalamide includes filtering or drying the terephthalamide. The combining, the heating, and the maintaining can be performed continuously in an extruder.

In a second general aspect, preparing a modified asphalt binder includes combining a terephthalamide produced by the first general aspect with an asphalt binder to yield a modified asphalt binder precursor and heating the modified asphalt binder precursor to yield a modified asphalt binder.

Implementations of the second general aspect can include the following feature. In some cases, a weight ratio of the asphalt binder to the terephthalamide is in a range of about 50:1 to about 10:1.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1A shows a flow-chart of a method of depolymerizing polyester material to yield a terephthalamide. FIG. 1B shows a flow-chart of a method of preparing a modified asphalt binder. FIG. 1C is a reaction schematic of an ethylene glycol mediated aminolysis reaction of polyethylene terephthalate (PET).

FIG. 2A shows attenuated total reflectance-Fourier transform infrared spectroscopy (ATR-FTIR) absorption spectra of PET and ethylene glycol. FIG. 2B shows structures of PET and ethylene glycol.

FIG. 3A shows ATR-FTIR absorption spectra of terephthalamide products N¹, N⁴-dioctylterephthalamide (OTP) (bottom), N¹, N⁴-didodecylterephthalamide (DTP) (mid) and N¹, N⁴-dihexadecylterephthalamide (HTP) (top) obtained from Set 1 reactions. FIG. 3B shows structures of PET, OTP, DTP, and HTP.

FIGS. 4A-4F show ATR-FTIR spectra of reaction Set 2 (2.1-2.6) between PET, hexadecylamine, and ethylene glycol at different reaction conditions. FIG. 4A shows an ATR-FTIR spectrum of reaction 2.1 of Set 2. FIG. 4B shows an ATR-FTIR spectrum of reaction 2.2 of Set 2. FIG. 4C shows an ATR-FTIR spectrum of reaction 2.3 of Set 2. FIG. 4D shows an ATR-FTIR spectrum of reaction 2.4 of Set 2. FIG. 4E shows an ATR-FTIR spectrum of reaction 2.5 of Set 2. FIG. 4F shows ATR-FTIR spectrum of reaction 2.6 of Set 2.

FIG. 5A shows an ATR-FTIR spectra of a Set 3 reaction obtained from an aminolysis reaction of PET in the presence (w/ethylene glycol (EG)) and absence (w/o EG) of ethylene glycol with N-octylamine (3.1 and 3.2). FIG. 5B shows an ATR-FTIR spectra of a Set 3 reaction obtained from an aminolysis reaction of PET in the presence (w/ethylene glycol (EG)) and absence (w/o EG) of ethylene glycol with dodecylamine (3.3 and 3.4). FIG. 5C shows an ATR-FTIR spectra of a Set 3 reaction obtained from an aminolysis reaction of PET in the presence (w/ethylene glycol (EG)) and absence (w/o EG) of ethylene glycol with hexadecylamine (3.5 and 3.6).

FIG. 6A shows gel permeation chromatography (GPC) elution traces of PET and terephthalamide product OTP. FIG. 6B shows GPC elution traces of PET and terephthalamide product DTP. FIG. 6C shows GPC elution traces of PET and terephthalamide product HTP.

FIG. 7 is a comparison of IR spectra of N¹, N⁴-dioctylterephthalamide generated from an ethylene glycol mediated aminolysis reaction of PET and microfiber (100% polyester) cloth.

FIG. 8 shows a reaction schematic of aminolysis reactions of PET with ethanolamine and octylamine.

FIG. 9 shows ATR-FTIR spectra of PET flakes (bottom) as well as synthesized N¹,N⁴-bis(2-hydroxyethyl)terephthalamide (HETP) (top) and OTP (middle) terephthalamides with characteristic peaks from functionalities highlighted.

FIG. 10A shows strain accumulation as a function of time during repeated loading cycles at 0.1 kPa. FIG. 10B shows strain accumulation as a function of time during repeated loading cycles at 3.2 kPa.

### DETAILED DESCRIPTION

This disclosure describes an ethylene glycol mediated aminolysis reaction of polyesters such as polyethylene terephthalate with a variety of long-chain primary linear alkylamines and branched alkylamines, at low reaction temperatures and short reaction times to yield terephthalamides. The terephthalamides are typically free or substantially free of impurities (e.g., oligomeric chains from unreacted PET). The reaction rate of polyethylene terephthalate (PET) aminolysis can be increased by using an appropriate mediator (e.g., ethylene glycol) to yield long-chain alkyl terephthalamides that can be used as value-added products for other processes. As used herein, “mediator” generally refers to an additive present in a reaction mixture that is not completely consumed in the reaction and can increase a reaction rate of the reaction under certain conditions. This disclosure shows the effectiveness of ethylene glycol as a mediator in the aminolysis of PET with primary long-chain alkylamines (e.g., butylamine (C4), amylamine (C5), hexylamine (C6), heptylamine (C7), octylamine (C8), nonylamine (C9), decylamine (C10), undecylamine (C11), dodecylamine (C12), tridecylamine (C13), tetradecylamine (C14), pentadecylamine (C15), hexadecylamine (C16), heptadecylamine (C17), octadecylamine (C18))) and branched alkylamines (e.g., isobutylamine, isopentylamine, (2-methylbutyl)amine, and 2-ethyl-1-hexylamine) to yield corresponding mono- and di-amides of terephthalic acids (e.g., terephthalamides). The aminolysis reactions are shown to go to 100% completion in the presence of an ethylene glycol mediator at low reaction temperatures (e.g., 125° C.) and short reaction times (e.g., 6 hours).

This depolymerization method can be effective with a mixed feed of polyester waste. The method also effectively removes dyes and impurities in the polyester products in the depolymerization step. This depolymerization can be achieved in a single reaction vessel and can be scaled to depolymerize large quantities (e.g., tons) of PET waste effectively. After depolymerization, there is no additional need for further purification or post processing of the terephthalamides. Methods described herein can be utilized to address the problem of recycling PET waste.

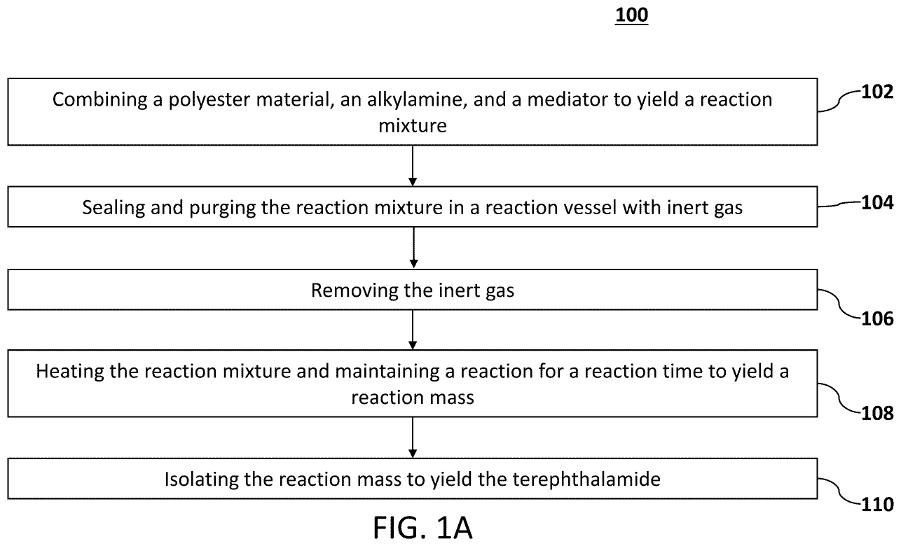

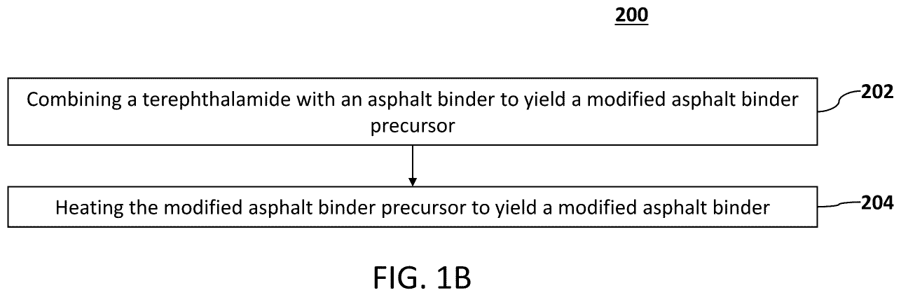

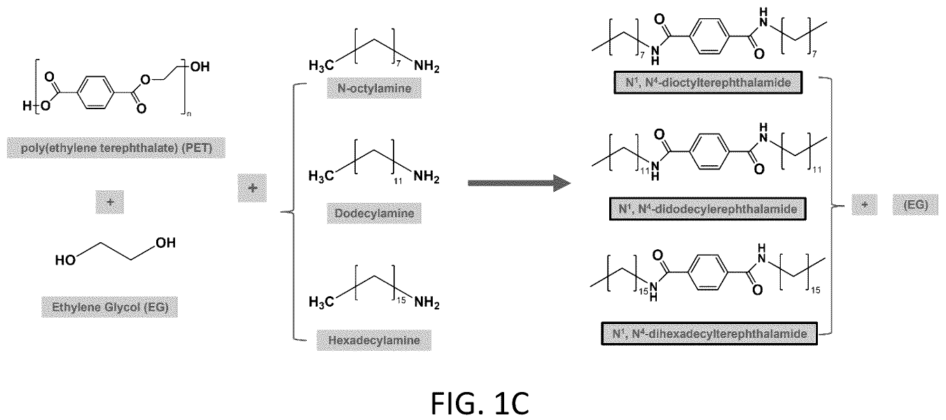

FIG. 1A depicts a flow chart showing operations of an example process 100 for depolymerizing polyester material to yield a terephthalamide. In 102, a polyester material, an alkylamine, and a mediator is combined to yield a reaction mixture. Examples of suitable polyester material include PET, polyethylene terephthalate-glycol, poly(trimethylene terephthalate), or any combination thereof. Examples of suitable alkylamine include butylamine, amylamine, hexylamine, heptylamine, octylamine, nonylamine, decylamine, undecylamine, dodecylamine, tridecylamine, tetradecylamine, pentadecylamine, hexadecylamine, heptadecylamine, octadecylamine, isobutylamine, isopentylamine, (2-methylbutyl)amine, 2-ethyl-1-hexylamine, or any combination thereof. An example of the mediator includes ethylene glycol. In some cases, a weight ratio of polyester:alkylamine:mediator in the reaction mixture is in a range of about 1:1:4 to 1:4:4 (e.g., 1:4:4). A molar ratio of polyester:alkylamine in the reaction mixture is typically in a range of 1:2 to 1:10 (e.g., 1:3.2, 1:4.2, or 1:6). In some cases, a molar ratio of polyester:mediator is in a range of 1:2 to 1:24 (e.g. 1:12.4).

In 104, the reaction mixture is placed in a reaction vessel, sealed, and purged with an inert gas. The inert gas is typically nitrogen. In 106, the inert gas is removed. In 108, the reaction mixture is heated and a reaction is maintained for a time to yield a reaction mass. The heating can include heating to a temperature in a range of 100° C. to 200° C. (e.g. 150° C.), or in a range of 120° C. to 130° C. (e.g., 125° C.). In some cases, the reaction time is between 1 hour and 72 hours (e.g., 2 hours, 6 hours, 24 hours, or 72 hours) or between 1 hour and 10 hours (e.g., 2 hours, 6 hours). The combining, the heating, and the maintaining is typically performed continuously in an extruder.

In 110, a reaction mass is isolated to yield the terephthalamide. Isolating the terephthalamide typically includes combining the reaction mass with a washing agent, filtering the reaction mass, drying the reaction mass, or any combination thereof. An example of a suitable washing agent includes acetone. The reaction mass is typically dried in a vacuum oven to yield the terephthalamide. Examples of terephthalamide include N¹, N⁴-dioctylterephthalamide (OTP), N¹, N⁴-didodecylterephthalamide (DTP), N¹, N⁴-dihexadecylterephthalamide (HTP), or any combination thereof.

This disclosure also describes synthetic terephthalamides derived from waste PET (e.g., N¹, N⁴-bis(2-hydroxyethyl)terephthalamide (HETP) and N¹, N⁴-(dioctyl)terephthalamide (OTP)), which can be used to modify an asphalt binder. The impact of these terephthalamides on the binder's elastic recovery under varying stress conditions and molecular interactions are assessed. The multiple-stress creep recovery test is used to evaluate shear-strain accumulation represented by percent shear strain during loading phases. Additionally, density functional theory (DFT) is used to analyze the intermolecular interactions between the modifiers and key binder components, assessing the modifiers' potential to be well-dispersed or to act as plasticizers within the binder mixture. A homogeneous binder microstructure with well-dispersed components (e.g., specifically asphaltenes and resins) is resistant to permanent deformation and minimizes the accumulation of strain.

The molecular features of HETP and OTP modifiers can lead to different outcomes in modified bitumen. The multiple-stress creep recovery tests show that OTP enhances the elasticity of the binder, supported by reduced strain accumulation during repeated loading cycles compared to that of unmodified asphalt binder. In contrast, HETP reduces the binder's elasticity, making it more susceptible to permanent deformation under stress. Density functional theory (DFT) calculations support these observations by demonstrating that OTP's dual non-polar eight-carbon arms and polar amide groups facilitate its distribution among asphaltene and resin molecules, forming stable asphaltene-OTP-resin complexes. The interaction energies of these complexes suggest that OTP can compete with a portion of asphaltene-asphaltene interactions, reducing asphaltene aggregation and contributing to the overall homogeneity and elasticity of the binder. On the other hand, similar stability or distribution within the binder matrix is not observed for HETP. HETP's challenge in overcoming the asphaltene-asphaltene interaction barrier can cause HETP to be bumped off strong binding sites, resulting in an uneven distribution and a less homogeneous binder structure, which is prone to deformation. Although HETP and OTP originate from the same recycled material, their molecular structures result in different effects on asphalt binder performance.

FIG. 1B depicts a flow chart showing operations of an example process 200 for preparing a modified asphalt binder. In 202, a synthesized terephthalamide is combined with an asphalt binder to yield a modified asphalt binder precursor. A weight ratio of the asphalt binder to the terephthalamide is typically in a range of about 50:1 to about 10:1 (e.g., 20:1). In 204, the modified asphalt binder precursor is heated to yield a modified asphalt binder. Heating typically includes heating to a temperature of 100° C. to 150° C. (e.g. 130° C.).

### EXAMPLES

### Example 1. Aminolysis Reactions

A representative experiment for the aminolysis reaction was conducted as follows. Commercial PET bottles were chopped into tiny pieces for the reaction and were subsequently loaded into a 50 mL round bottom flask with a magnetic stir bar. Long-chain alkylamines (e.g., N-octylamine, dodecylamine, and hexadecylamine) were then added to the round bottom flask along with ethylene glycol. The round bottom flask was then sealed and purged with nitrogen for 15 minutes along with constant stirring. The nitrogen was removed, and then the round bottom flask was lowered in an oil bath maintained between 125° C. to 150° C. The reaction time was varied between 6 hours and 72 hours until complete conversion was achieved. After 24 hours, the obtained reaction mass was mixed with an excess of acetone to remove any unreacted amines and ethylene glycol. The reaction mass was then filtered and isolated. The obtained product (e.g., terephthalamide) was dried in vacuum oven for 24 hours at 80° C. to ensure complete removal of acetone. FIG. 1C shows the reaction schematic of an ethylene glycol mediated aminolysis reaction of PET. Using the above protocol, PET depolymerization reactions were performed with N-octylamine, dodecylamine, and hexadecylamine in the presence and absence of ethylene glycol to yield N¹, N⁴-dioctylterephthalamide (OTP), N¹, N⁴-didodecylterephthalamide (DTP), and N¹, N⁴-dihexadecylterephthalamide (HTP), respectively.

The aminolysis reactions mentioned above were conducted in different configurations with respect to the reaction conditions to evaluate the most optimized reaction pathway. As shown in Table 1, 3 sets of reaction conditions were assessed. Set 1, a preliminary data set, included 3 reactions between PET and 3 primary amines (e.g., N-octylamine (reaction 1.1), dodecylamine (reaction 1.2) and hexadecylamine (reaction 1.3)) in the presence of ethylene glycol with a weight ratio of 1:1:4 (PET:Amine:ethylene glycol (EG)) at 125° C. for 24 hours, as shown in Table 1. In Set 2, hexadecylamine was reacted with PET and ethylene glycol at different reaction temperatures, reactions times, and weight ratios, as shown in Table 2. Set 3 reactions were performed between PET and the 3 primary amines in the presence and absence of ethylene glycol to test the efficacy of ethylene glycol as a mediator for the reaction, as shown in Table 3. The products from each of these reactions (Set 1-Set 3) were qualitatively evaluated with attenuated total reflectance-Fourier transform infrared spectroscopy (ATR-FTIR) analysis for confirmation of the resulting terephthalamide functionality from the reaction.

TABLE 1
Set 1 reaction conditions for optimization of the aminolysis reaction
between PET, 3 primary long-chain amines, and ethylene glycol.

| | Weight Ratio | Molar Ratio | | | | |
|---|---|---|---|---|---|---|
| Reaction | (PET:Amine:EG) | | | | | Ethylene |
| Set 1 | g basis | PET | Octylamine | Dodecylamine | Hexadecylamine | Glycol |
| 1.1 | 1:1:4 | 1.0 | 1.5 | | | 12.4 |
| 1.2 | 1:1:4 | 1.0 | | 1.0 | | 12.4 |
| 1.3 | 1:1:4 | 1.0 | | | 0.8 | 12.4 |

TABLE 2
Set 2 reaction conditions for aminolysis reaction
between PET, hexadecylamine, and ethylene glycol.

| | PET:Amine:EG | | | |
|---|---|---|---|---|
| Set 2 | Weight | Molar | Reaction Time | Reaction |
| Reaction | Ratio | ratio | (h) | Temperature |
| 2.1 | 1:2:4 | 1:1.6:12.4 | 24 | 125° C. |
| 2.2 | 1:1:8 | 1:0.8:24.8 | 24 | 125° C. |
| 2.3 | 1:2:8 | 1:1.6:24.8 | 24 | 125° C. |
| 2.4 | 1:2:4 | 1:1.6:12.4 | 24 | 150° C. |
| 2.5 | 1:1:4 | 1:0.8:12.4 | 24 | 150° C. |
| 2.6 | 1:1:4 | 1:0.8:12.4 | 72 | 125° C. |

TABLE 3
Set 3 conditions for the aminolysis reaction
between PET and 3 primary long-chain amines
in the presence and absence of ethylene glycol.

| | | | PET:Amine:EG/PET:Amine | |
|---|---|---|---|---|
| Set 3 | | Is Ethylene | Weight | Molar |
| Reaction | Amine Used | Glycol used? | Ratio | Ratio |
| 3.1 | Octylamine | Yes | 1:4:4 | 1:6:12.4 |
| 3.2 | | No | 1:4:0 | 1:6 |
| 3.3 | Dodecylamine | Yes | 1:4:4 | 1:4.2:12.4 |
| 3.4 | | No | 1:4:0 | 1:4.2 |
| 3.5 | Hexadecyl- | Yes | 1:4:4 | 1:3.2:12.4 |
| 3.6 | amine | No | 1:4:0 | 1:3.2 |

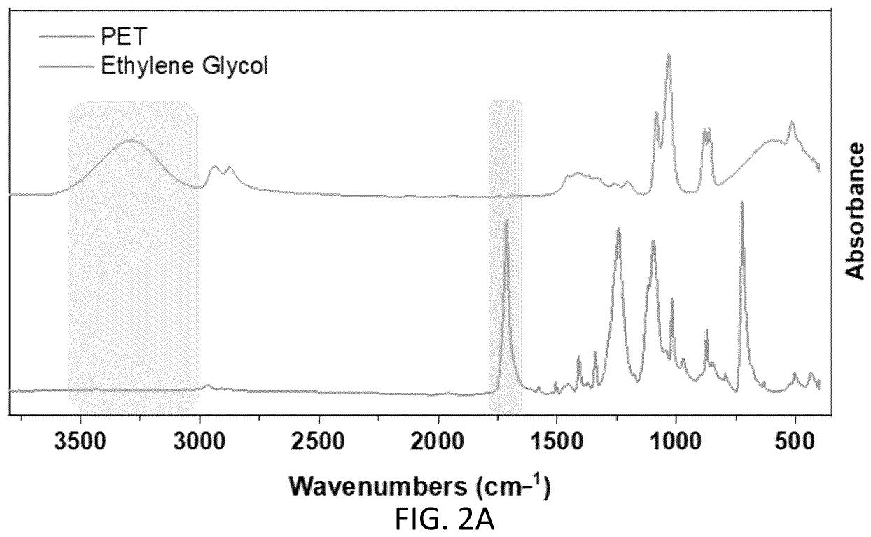

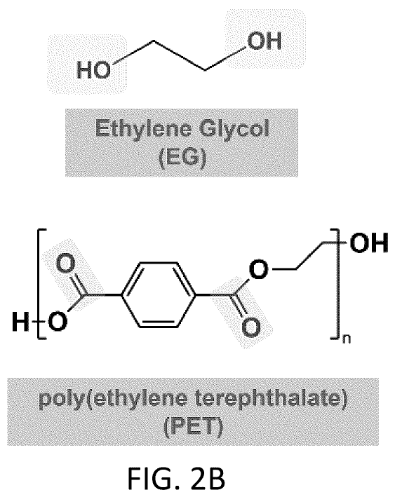

ATR-FTIR analysis was utilized to identify changes in the functional groups of reactants and products before and after the reaction. For reference, example absorption FTIR spectra of PET and ethylene glycol are shown in FIG. 2A. The highlighted peak in each spectrum identifies the characteristic functional groups of PET and ethylene glycol. For PET, the strong absorption band between wavenumbers 1715 cm⁻¹ and 1740 cm⁻¹ corresponds to a carbonyl (C═O) stretch of the ester peak in the PET structure. Likewise, the strong and broad stretch between 3200 cm⁻¹ and 3500 cm⁻¹ corresponds to the hydroxyl (—OH) functionality in the ethylene glycol structure. These peaks from PET and ethylene glycol were used as reference for any left-over reactants in the terephthalamide spectra to confirm complete conversion.

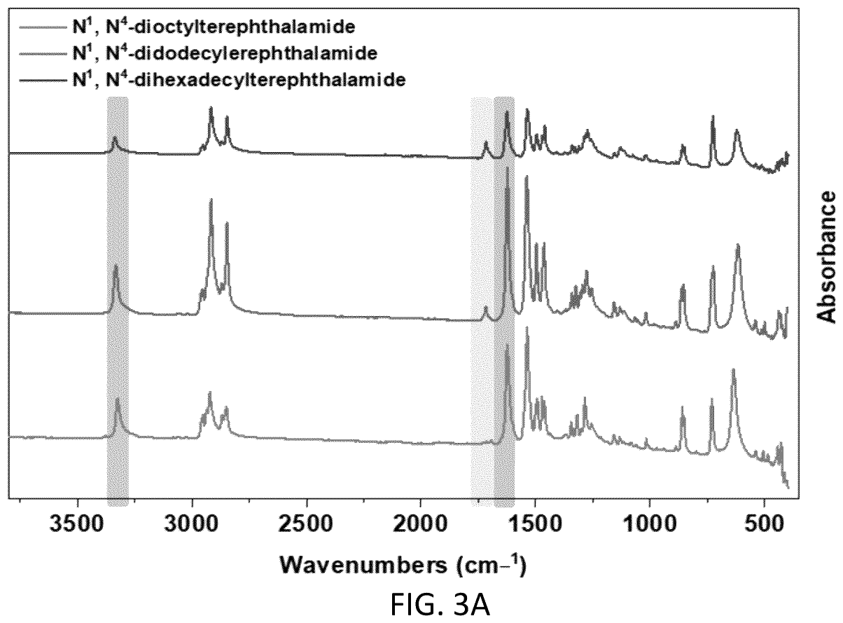

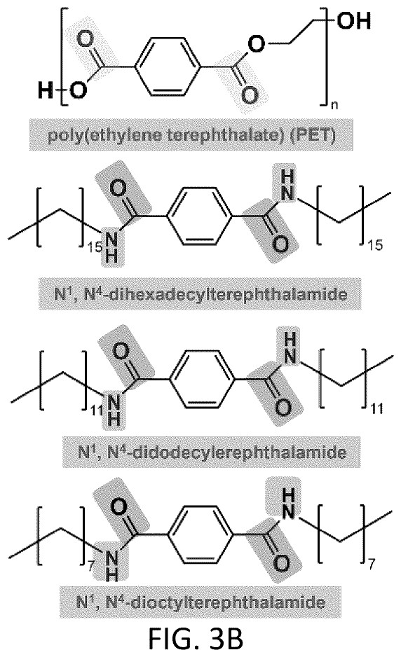

For Set 1 reactions, the ATR-FTIR spectra of terephthalamide products OTP (bottom), DTP (mid), and HTP (top) are shown in FIG. 3A. In Set 1, the resulting terephthalamide functionality was identified by the characteristic amide (—CONH—) linkage where carbonyl peak stretching (—C═O) and secondary amide (—NH—) peak were observed near 1630 cm⁻¹ and 3300 cm⁻¹, respectively. As shown in FIGS. 3A and 3B, the strong carbonyl stretch (—C═O) at 1715 cm⁻¹ from the ester disappeared in the OTP product spectrum and a new peak appeared at 1630 cm⁻¹ and 3300 cm⁻¹, corresponding to an amide linkage. This confirmed depolymerization of terephthalates to terephthalamides. In one example, in the DTP and HTP IR spectra, carbonyl peaks at 1715 cm⁻¹ (ester) and 1630 cm⁻¹ (amide) co-existed. This suggested that reactions (e.g., 1.2 and 1.3) did not go to completion, resulting in leftover PET polymer in the reaction mixture.

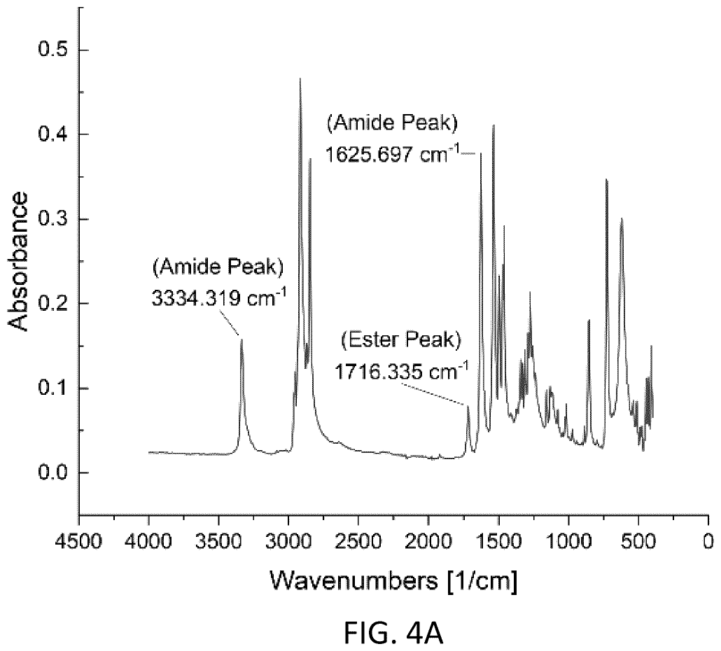

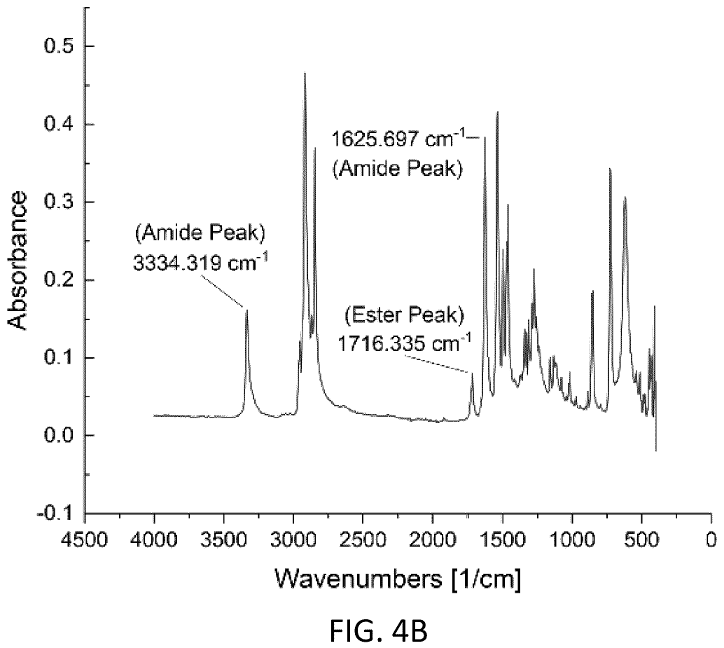

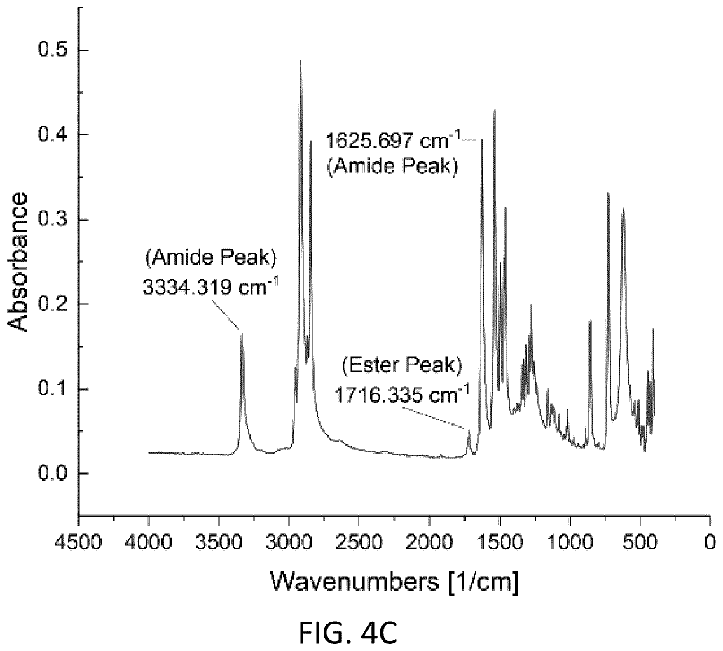

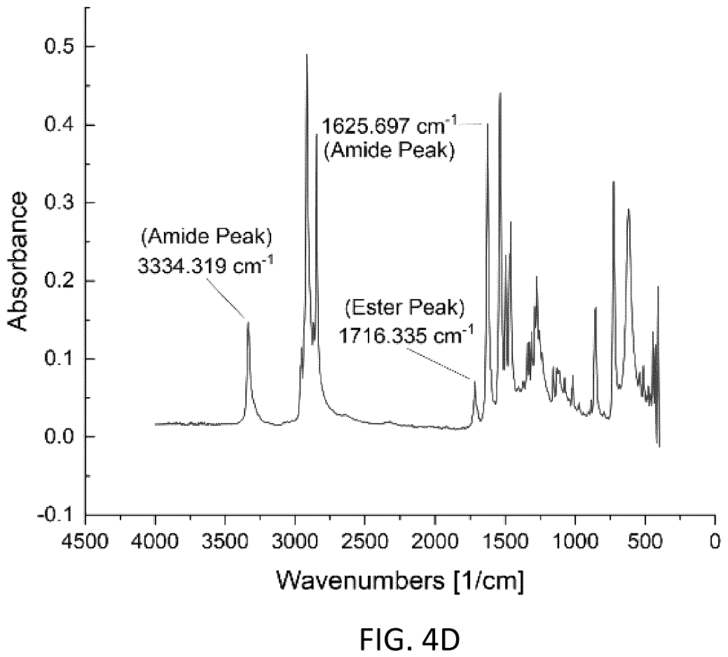

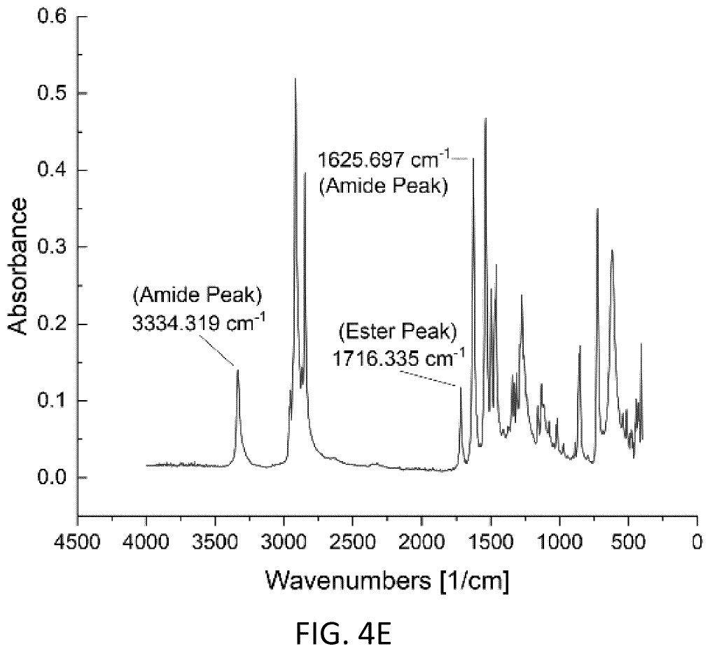

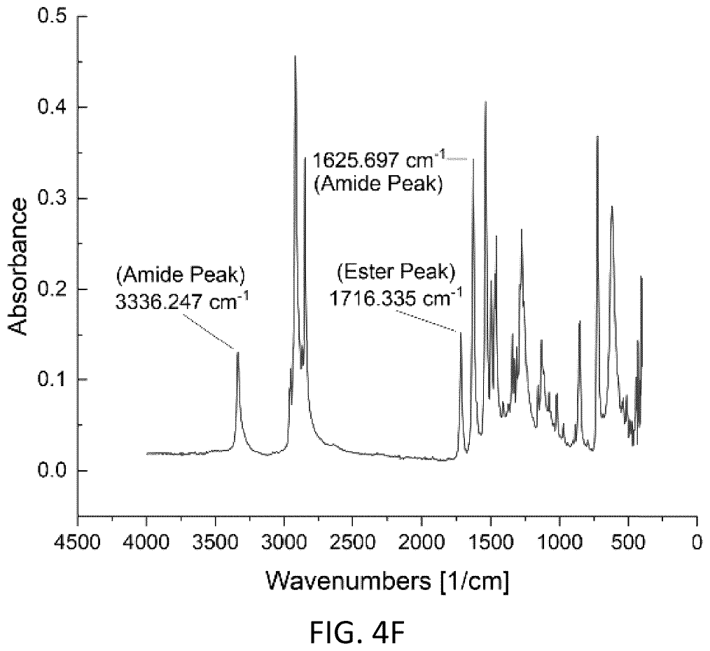

As shown in Table 2, Set 2 parameters such as temperature, time, and weight ratios were varied with hexadecylamine reactant to optimize the reaction conditions. FIGS. 4A-4F show ATR-FTIR spectra of reaction set 2 (2.1-2.6) listed in Table 2 between PET, hexadecylamine, and ethylene glycol with different reaction conditions. The IR spectra shown in FIGS. 4A-4F show that these reactions did not go to completion, based on the signal from the ester peaks from the presence of residual PET. As shown in FIGS. 4A-4F, the carbonyl peaks from ester and amide functionalities co-exist along with the secondary amide peaks at 3334 cm⁻¹. Although conversion of terephthalates to terephthalamides was observed, the presence of carbonyls suggested PET remnants in the reaction mixtures. Reactions 2.1 and 2.4 performed at the same weight ratios at 125° C. and 150° C. respectively, suggested that there is a need to further increase the primary amine reactant concentration in the reaction instead of the reaction temperature.

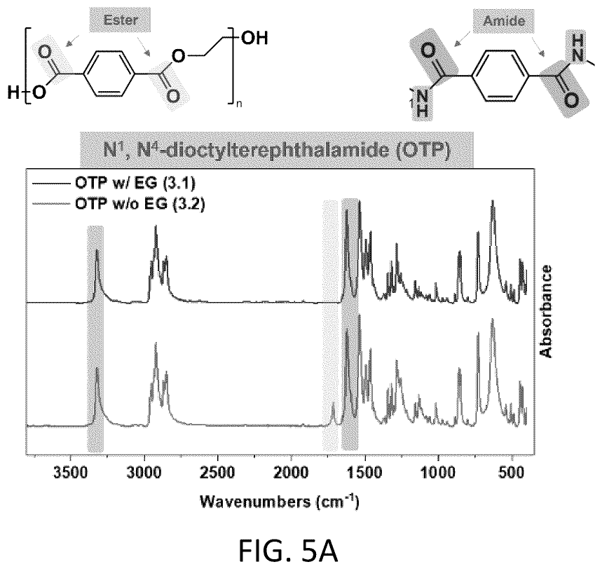

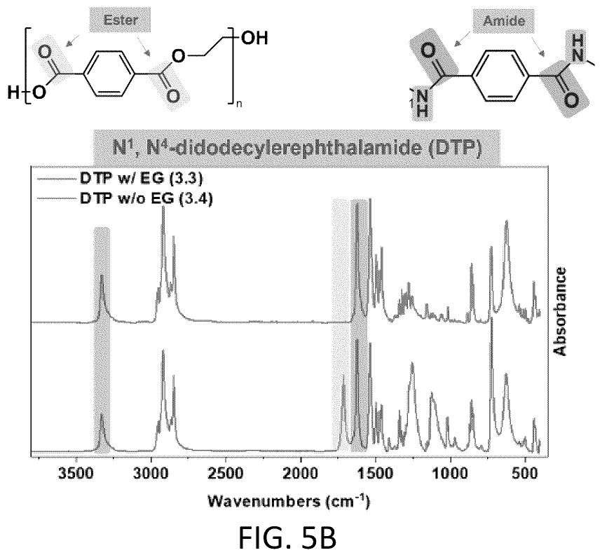

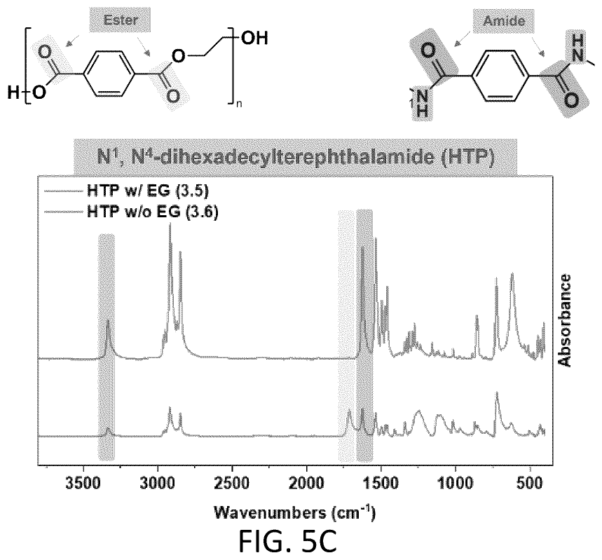

FIGS. 5A-5C show ATR-FTIR spectra of Set 3 reactions obtained from aminolysis reaction of PET in the presence (w/EG) and absence (w/o EG) of ethylene glycol with N-octylamine (3.1 and 3.2), dodecylamine (3.3 and 3.4) and hexadecylamine (3.5 and 3.6).

The effectiveness of ethylene glycol as a suitable mediator for the aminolysis reaction of PET was tested in Set 3 reactions along with increasing the weight ratio of the amines to 1:4:4 (PET:Amine:EG) at 125° C. In FIGS. 5A-5C, spectra of reactions 3.1, 3.3, and 3.5 corresponding to products OTP, DTP, and HTP, respectively, suggested that all the PET has depolymerized to terephthalamides with the addition of ethylene glycol to the reaction. This was noted by the absence of a carbonyl peak at 1715 cm⁻¹ from PET. In the absence of ethylene glycol (3.2, 3.4, and 3.6) there was a partial conversion of the PET to terephthalamides, and the ester peak was observed along with the amide peaks in the IR spectra. This suggested that ethylene glycol effectively aided in accelerating this reaction forward to achieve complete depolymerization of PET.

Molecular weight analysis with gel permeation chromatography (GPC) was performed on neat PET flakes, and OTP, DTP, and HTP products. This GPC analysis was performed using a TOSOH EcoSec HLC-8321 High Temperature GPC System. The entire system was set to 110° C. The mobile phase used was 2-chlorophenol. Sample columns were set to an operating flow rate of 1.0 mL/min and the reference column was set to an operating flow rate of 0.5 mL/min. A differential refractive index (dRI) detector was used. A Mark Houwink adjusted polystyrene calibration curve was used to determine molecular weight values. Mark Houwink values for polystyrene were K=12.1×10⁻⁵ and a=0.707. Mark Houwink values for PET were K=96.3×10⁻⁵ and a=0.658.

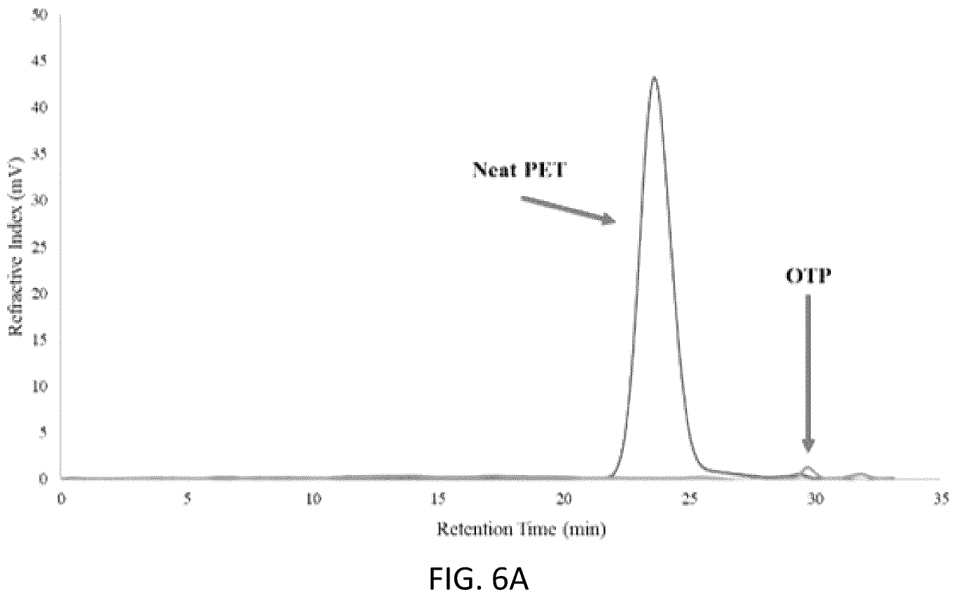

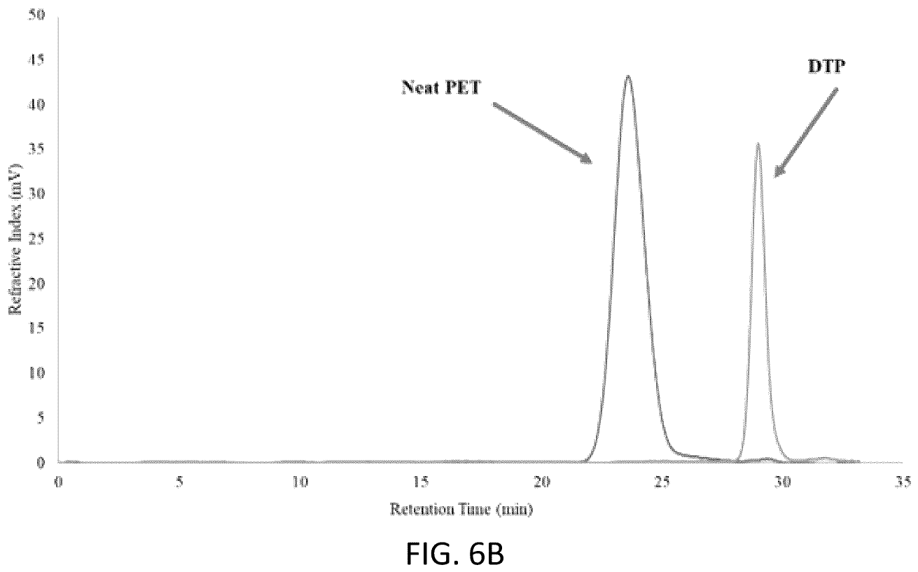

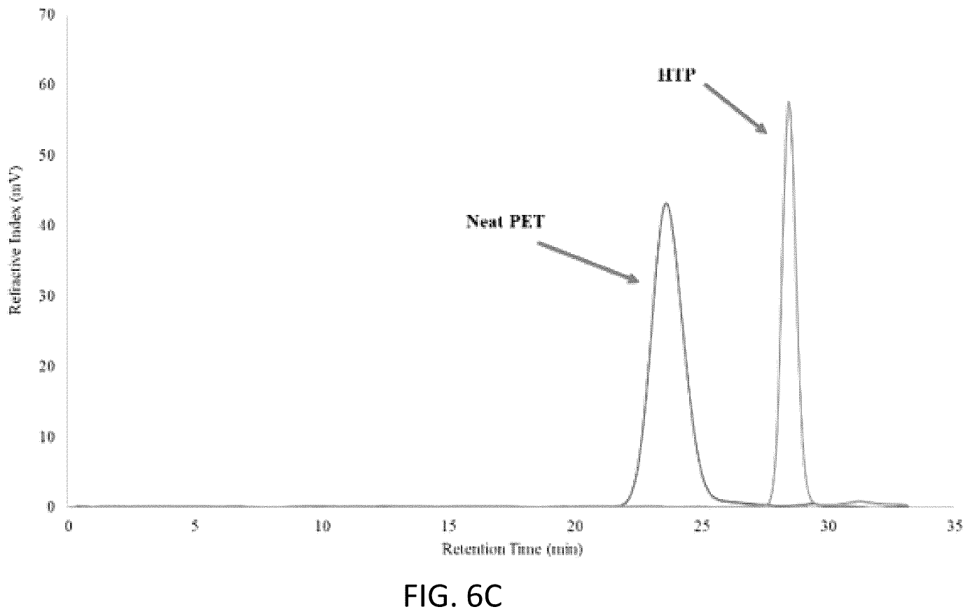

FIGS. 6A, 6B, and 6C show GPC elution traces of PET and terephthalamide products from aminolysis reaction. FIG. 6A shows GPC elution traces of PET and OTP, FIG. 6B shows GPC elution traces of PET and DTP, and FIG. 6C shows GPC elution traces of PET and HTP. The GPC elution traces of terephthalamides from Set 3 reactions (3.1, 3.3 and 3.5) are shown in FIGS. 6A, 6B, and 6C and overlaid with elution profile of PET. Table 4 summarizes the molecular weight analysis obtained from the GPC elution profile. The terephthalamides, OTP, DTP, and HTP, obtained from the aminolysis reaction with PET in presence of ethylene glycol showed one distinct peak at elution times 29.7 minutes, 28.9 minutes, and 28.4 minutes, respectively in the GPC trace. Furthermore, the obtained products OTP, DTP, and HTP, were monodispersed with an estimated number average molecular weight (MW) of 132 Da, 207 Da and 301 Da, respectively. In comparison, neat PET utilized in these reactions has a high MW of 21000 Da (Number Average MW). This dataset showed that utilizing ethylene glycol as a mediator was effective in depolymerizing PET and yielded impurity-free terephthalamides with different chain lengths.

TABLE 4
Summary of GPC analysis of the products from the ethylene
glycol mediated aminolysis of PET. The molecular weight
(MW), number average molecular weight (Mn), polydispersity
index (PDI), and retention times are listed.

| | | | | Retention |
|---|---|---|---|---|
| | Mw | Mn | | Time |
| Sample Name | (Da) | (Da) | PDI | (min) |
| Neat PET | 41,028 | 21,003 | 1.953 | 23.6 |
| N¹,N⁴-dioctylterephthalamide | 136 | 132 | 1.023 | 29.7 |
| (OTP) | | | | |
| N¹,N⁴-didodecylterephthalamide | 215 | 207 | 1.043 | 28.9 |
| (DTP) | | | | |
| N¹,N⁴-dihexadecylterephthalamide | 311 | 301 | 1.034 | 28.4 |
| (HTP) | | | | |

In one example, an in-situ FTIR analysis was performed by inserting an IR probe to the ethylene glycol mediated aminolysis reaction of PET. PET was reacted with hexadecylamine and ethylene glycol (e.g., 1:4:4) and IR spectra were collected every 3 minutes for a 24-hour reaction time. A 3D surface plot (absorbance vs. wavenumber vs. reaction time) and the corresponding heat map generated by collating in-situ IR spectroscopy data from the ethylene glycol mediated aminolysis reaction (hexadecylamine) of PET was assessed. The map showed at t>2.5 hours, the carbonyl peak (—C═O) from the amide linkage (—CONH—) emerged between 1630 cm⁻¹ and 1650 cm⁻¹ wavenumbers. The peak intensified between 2.5 hours<t<5.1 hours and then lowered in intensity signifying that aminolysis reaction took place between this time frame. Thus, with the introduction of ethylene glycol in the aminolysis reaction, complete depolymerization of PET was achieved in less than 6 hours and at a moderately lower reaction temperature of 125° C.

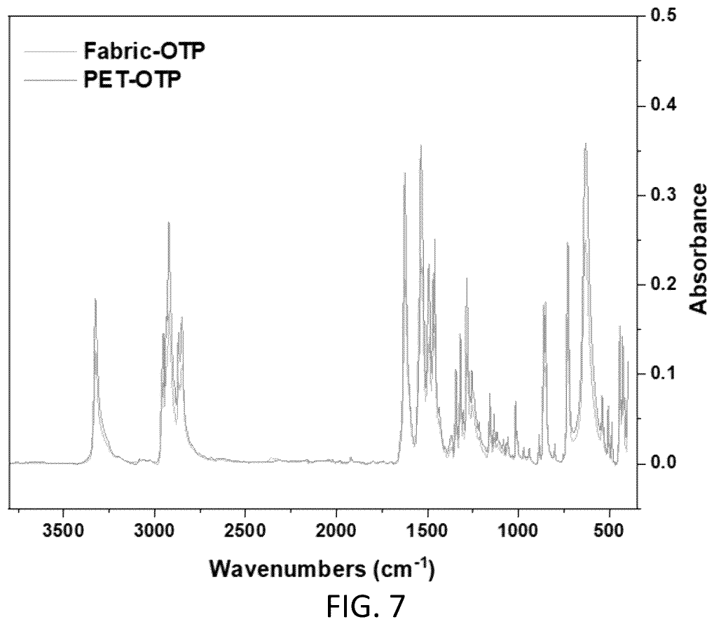

The depolymerization experiments for PET waste plastics were further extended to commercial fabrics (e.g., 100% polyester microfiber cloth). A preliminary experiment was conducted where the commercially available (e.g., 100% polyester) microfiber cloth was reacted with N-octylamine and ethylene glycol in a 1:4:4 ratio at 125° C. for 6 hours to yield OTP from aminolysis. The end product yielded a white powder, similar to the reaction with PET bottles, suggesting that the depolymerization was successful. In one example, the ethylene glycol mediated aminolysis reaction was also effective in removing the dye from the microfiber cloth. The IR spectrum of the formed N¹, N⁴-dioctylterephthalamide (Fabric-OTP) from this reaction was compared to the IR spectrum of the N¹, N⁴-dioctylterephthalamide obtained from PET waste bottle reaction (PET-OTP), as shown in FIG. 7. Here, the appearance of amide peaks (—C═O and —NH—) and the disappearance of carbonyl from the ester functionality suggested the presence of a similar product from the two reactions.

### Example 2. PET-Derived Modifiers for Terephthalamide Asphalt Binders

The asphalt binder used was a PG 70-10 grade obtained from Hollyfrontier Sinclair Corporation. This asphalt binder served as the base binder for producing polymer-modified binders. Ethanolamine (ACS reagent, 99%) and N-octylamine (ACS reagent, 99.0%) were purchased from Sigma Aldrich and used as received. Ethylene glycol (99.0%) and acetone (99.9%) were purchased from VWR Inc.

PET beverage bottles were sourced from a nearby recycling bin, washed thoroughly with tap water, and then allowed to dry. Labels and any other plastic components were removed, and any remaining adhesive was cleaned off using hexanes. The PET bottles were cut into flakes of approximately 5 mm×5 mm which was used to synthesize two terephthalic amides: N¹,N⁴-bis(2-hydroxyethyl)terephthalamide (HETP) and OTP.

HETP was synthesized using reference protocols. In one example, PET (0.05 mol) flakes were mixed with an excess of ethanolamine in a 2-neck flask equipped with a magnetic stirrer and nitrogen outlet. Excess ethanolamine (3 eq) was added, and the mixture was heated to a temperature in a range of 130° C. to 140° C., during which the solution became less viscous as ethylene glycol was generated. As the PET flakes were consumed, the product began to form and precipitated out of the solution. To maintain homogeneity, the temperature was increased to 150° C. After 2 hours of heating, the reaction was stopped by cooling the mixture to room temperature and the resulting precipitate was suspended in excess of acetone. The HETP product was isolated by filtration followed with an additional acetone wash and subsequent isolation.

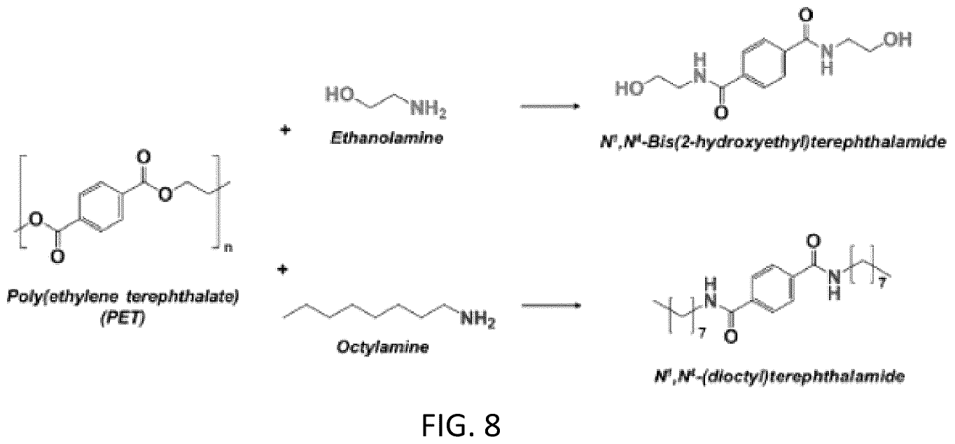

PET flakes (0.05 mol) were suspended in a 2-neck flask equipped with a magnetic stirrer and a nitrogen flow. An excess of N-octylamine (0.03 mol) was added to suspend PET flakes in the solution and ensure proper mixing. Ethylene glycol (1.2 mol) was also added as a co-solvent to aid in accelerating the aminolysis reaction. Next, the reaction mixture was stirred under an inert nitrogen atmosphere for 5 minutes, followed by heating for 6 hours at 125° C. As the reaction proceeded, the OTP precipitated out of the solution, forming a phase cake layer on top of the reaction mixture. After 6 hours, the reaction mass was washed with an excess of acetone and isolated by filtration. An additional acetone wash was performed to remove any excess amines and glycols from the reaction mass. The final isolated OTP product had a white appearance. FIG. 8 shows the aminolysis of PET and molecular structure of the HETP and OTP.

The terephthalamides were qualitatively characterized using FTIR. A Thermo Scientific™ Nicolet™ iS50 FTIR Spectrometer was used for FTIR measurements of samples. The iS50 ATR module accessory with a diamond crystal was utilized as the stage for all samples. Measurements of each sample were done at a resolution of 4 cm⁻¹ resolution and an average of over 32 scans after collecting the background.

To prepare the modified binders, synthetic terephthalamides were mixed with asphalt binders at a concentration of 5% weight percent compared to the original asphalt binder. The mixing was carried out at a temperature of 130° C. using an industrial mixer at 400 rpm for 5 min.

The multiple-stress creep recovery test was conducted using an Anton Paar dynamic shear rheometer with an 8-mm spindle. The test evaluated the rheological properties of asphalt binders under intermediate-temperature conditions (e.g., 45° C.) when subjected to repeated cycles of loading and rest. The multiple-stress creep recovery test procedure involved applying stress levels of 0.1 kPa and 3.2 kPa in succession to a sample of asphalt binder. Each stress level underwent 10 cycles of creep and recovery, where each cycle includes a 1-second creep (load application) period followed by a 9-second recovery (load removal) period. The test included 20 cycles with a total test duration of 200 seconds performed at two stress levels of 0.1 kPa and 3.2 kPa. Shear-strain accumulation represented by percent shear strain during loading phases graphically showed the deformation and recoverable-strain characteristics of the asphalt binder, indicating how much permanent deformation occurred during loading or how effectively the binder recovered during the rest period after each loading cycle.

DFT calculations were performed to explore the interactions between the two terephthalamides, HETP and OTP, and bitumen components. Geometry optimizations to compare these intermolecular interactions were conducted using non-periodic DFT calculations with the Perdew-Burke-Ernzerhof (PBE) afunctional and generalized gradient approximation (GGA). To account for dispersion interactions between the fragments, dispersion-corrected DFT (PBE-D) calculations were employed using Grimme's correction. The calculations were carried out using the Dmol3 module24 in the Accelrys Materials Studio program package (version 7.0). An all-electron double-numerical basis set with polarization (DNP) was used, with convergence criteria set at 1.0×10⁻⁵ hartree for energy, 2.0×10⁻³ hartree/Å for maximum force, and 5.0×10⁻³ Å for displacement. Interaction energies (Eᵢₙₜ) for the intermolecular interactions were calculated using Equation 1.

$${E}_{int} = {E}_{complex} - ( \sum {E}_{fragments} ) \qquad ( 1 )$$

where E_{complex} represents the total energy of the HETP-bitumen and OTP-bitumen complexes, and ΣE_{fragments} is the sum of the energies of the fragments in the optimized complex.

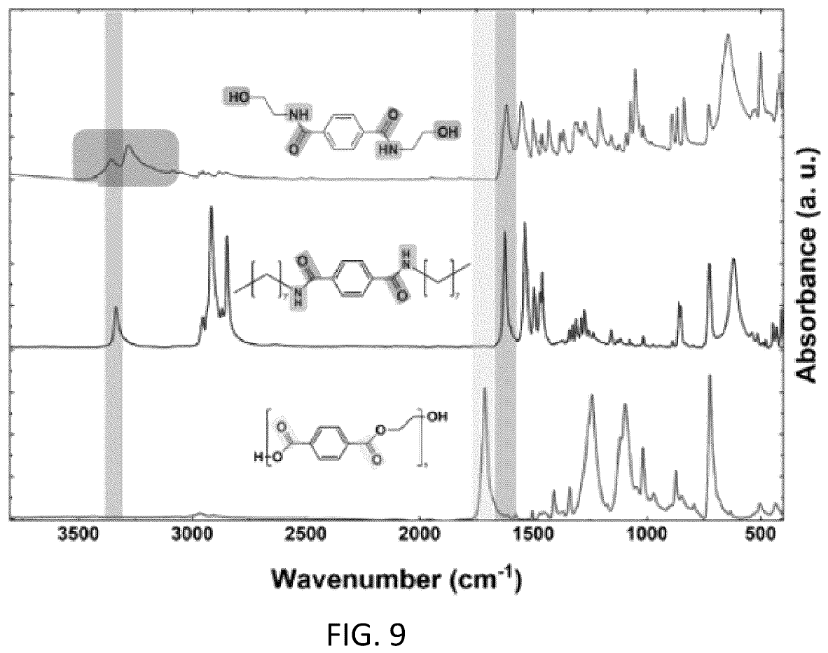

FTIR analysis was used to qualitatively characterize the terephthalamides synthesized and to observe the changes in the functional groups after the aminolytic depolymerization of the PET. Referring to FIG. 9, the PET IR spectrum showed a strong absorption band between wavenumbers 1715 cm⁻¹ and 1740 cm⁻¹, corresponding to the carbonyl (C═O) of the ester peak. This characteristic absorption band of PET was used as an indicator to assess the complete conversion of carbonyl esters to terephthalamide functionalities. For OTP, a new absorption peak appeared at 1630 cm⁻¹ and 3300 cm⁻¹, corresponding to the carbonyl stretching (C═O) and secondary amine (—NH—) stretching, respectively from the resulting terephthalamide (—CONH—) functional group. Similarly, HETP showed both these absorption bands along with an additional broad hydroxyl (—OH) band between 3200 cm⁻¹ and 3500 cm⁻¹ associated to the ethoxy groups bonded to the amide linkage. The identification of these absorption bands suggested the successful conversion of terephthalates to terephthalamides. Furthermore, neither OTP nor HETP showed the presence of carbonyl absorption bands between 1715 cm⁻¹ and 1740 cm⁻¹ from the ester groups, further suggesting full conversion in both synthetic protocols performed.

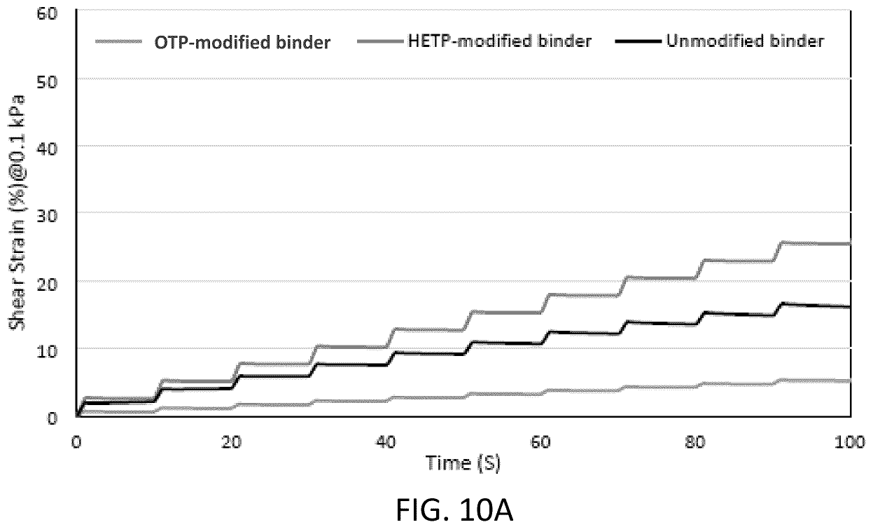

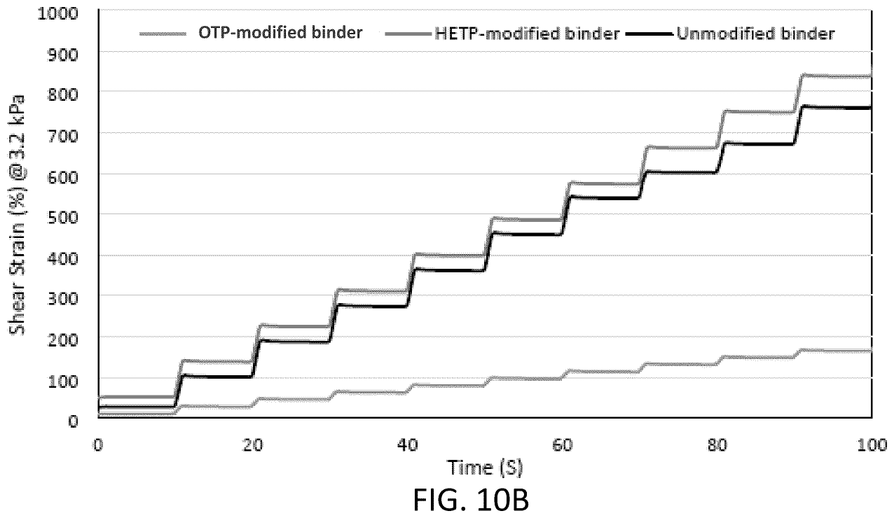

The multiple-stress creep recovery test is a method for assessing the resistance to permeant deformation as represented by accumulated strain (ASTM D7405-1) during repeated loading asphalt binders. By applying repeated stress to binders and evaluating their recovery capabilities, the test provided insight into the material's performance in real-world scenarios. The multiple-stress creep recovery test simulated the conditions that asphalt binders face in the field, particularly concerning repeated traffic loading and high temperatures. To understand the total deformation behavior of the samples of modified binders and unmodified binders, the percent shear strain was calculated as shown in FIGS. 10A and 10B. FIG. 10A shows the percent shear strain at 0.1 kPa, and FIG. 10B shows the percent shear strain at 3.2 kPa. The OTP-modified binder exhibited a lower percent shear strain relative to the unmodified binder whereas the HETP-modified binder showed a higher percent shear strain than the unmodified binder. These results suggested that the HETP-modified binder is susceptible to permanent deformation, highlighting its limited applicability without further modification. In contrast, the OTP-modified binder showed improvement across various stress and loading conditions.

Although both modifiers were derived from the same PET source using identical PET flake sizes and nearly identical synthetic processes, the difference between them can be due at least in part to in their molecular structure and distinct molecular properties. The HETP has short, polar arms with a free hydroxyl group at each end, while the OTP has longer hydrophobic arms. Both compounds have the terephthalamide group in the center of the molecule, as shown in FIG. 8. These properties at the atomistic and molecular levels can influence the intermolecular interactions with the binder, suggesting a subsequent effect on the microstructure. The microstructure of the binder can impact other properties, including the viscoelastic behavior. To provide more insight of the variational effects of HETP and OTP on the intermolecular interactions within the binder, DFT calculations were used.

The microstructure of bitumen can be used to determine its elastic properties such as creep recovery and strain accumulation. Bitumen, a complex material composed of asphaltenes, resins, saturates, and aromatics, relied heavily on the distribution and interaction of these components to define its microstructure. When asphaltenes are well dispersed, they can contribute to the overall stiffness of bitumen, improving its elastic recovery. Asphaltene molecules tend to aggregate due at least in part to polar-polar interactions and π-π stacking. This phenomenon can lead to asphaltene precipitation in crude oil reservoirs and contribute to inhomogeneity in bitumen mixtures. Resins can play role in stabilizing asphaltenes by acting as a dispersant. When modifiers are added to bitumen mixtures, these modifiers can not only disperse well in the bitumen matrix but also contribute to the overall stability of the mixture. This included assisting in the dispersion of asphaltenes and preventing their aggregation. Heterogeneous regions in bitumen mixture, having agglomerations of asphaltenes, can act as weak points, leading to greater strain accumulation under repeated loading. The formation of large asphaltene clusters, due at least in part to poor dispersion, can cause the material to behave more plastically, reducing its elastic property. Bitumen's response to stress was both elastic (e.g., recoverable) and viscous (e.g., permanent), with the microstructure influencing this balance. Modifiers and additives can be used to enhance the microstructure of bitumen, promoting better dispersion and homogeneity. These modifications can lead to a more interconnected network within the bitumen that resists deformation, improving compatibility among components, and resulting in better creep recovery and reduced strain accumulation.

Another factor in achieving a well-structured microstructure was the polarity of the medium. The balance between polar and non-polar components in the binder matrix contributed to maintaining the stability and homogeneity of the bitumen. The molecular structure of modifiers can allow them to bind and disperse effectively in the bitumen matrix, whether through covalent bonding, hydrogen bonding, charge transfer, π-π interactions, or dispersion forces. At least in part because bitumen contained highly non-polar components, modifiers with polar functional groups disperse well in the matrix, specifically by bridging between the bitumen components (e.g., similar to a surfactant effect). Therefore, increased intermolecular interaction between the modifier and different components of asphalt binder, prevention of agglomerations, and maintaining a polar/non-polar balance in the binder matrix were considered for developing a well-structured microstructure in bitumen.

DFT was used to investigate the intermolecular interactions between modifiers and candidate binder components. HETP and OTP are bis-phthalamide molecules, each including a benzene ring with two amide linkers to the side chains. HETP features four polar functional groups-two amide groups and two hydroxyl groups-separated by two carbon (e.g., ethyl) groups. In contrast, OTP contains two polar amide groups located between the benzene ring and a non-polar side chain with eight carbons (e.g., octyl groups); OTP lacked hydroxyl groups in its molecular structure, as shown in FIG. 8.

Due at least in part to its four polar functional groups and fewer carbon atoms, HETP was a highly polar molecule, so HETP could form strong polar-polar attractions with the polar molecules in the binder. In contrast, OTP, with both polar regions and non-polar regions (e.g., a surfactant-like structure), had the potential to interact with both the polar components and the non-polar components of the binder. Asphaltene and resin molecules, which possess both polar and non-polar groups, were more likely to interact with these two bis-phthalamide modifiers. The π-π stacking interactions between fused aromatic rings in asphaltene played a role in asphaltene agglomeration and the uneven distribution of hydrocarbons in bitumen. An asphaltene structure featuring a condensed core fused aromatic ring and a polar pyrrole ring was selected for assessment. Additionally, a quinoline resin was chosen for molecular modeling.

First, the interactions between each HETP and OTP molecule with the asphaltene structure were calculated. The results showed that both HETP and OTP formed hydrogen bonds with the acidic hydrogen of the asphaltene (N—H) through the carbonyl oxygen (C═O) of their amide functional groups (—CONH—). The interaction energies for the HETP-asphaltene and OTP-asphaltene complexes were found to be −36.5 kcal/mol and −46.7 kcal/mol, respectively. The higher interaction energy for OTP-asphaltene was attributed at least to its ability to form additional interactions, such as charge-transfer and dispersion interactions, between its non-polar arms and the non-polar aromatic and side alkyl chains of asphaltene.

Then, the HETP-asphaltene and OTP-asphaltene interaction energies were compared to asphaltene-asphaltene interaction energies. Asphaltene-asphaltene interactions were evaluated using the same computational method. Various orientations and conformations were considered, leading to the identification of two interacting complexes with high interaction energies. In the complex with the highest interaction energy (−75.3 kcal/mol), the polar N—H groups were oriented close to each other, enhancing polar electrostatic attractions, while the fused aromatic rings were aligned parallel to each other, maximizing π-π stacking. Another complex, where the polar N—H groups were positioned far apart and π-π stacking was the dominant attraction force between the molecular fragments, had an interaction energy of −52.5 kcal/mol.

This suggested that the interaction energies of both HETP-asphaltene (−36.5 kcal/mol) and OTP-asphaltene (−46.7 kcal/mol) complexes were lower than those of the asphaltene-asphaltene complexes.

In the next step, tertiary complexes, HETP-asphaltene-resin and OTP-asphaltene-resin, were simulated. The structures of these tertiary complexes were optimized, and the interaction energies between the fragments in the complexes were evaluated. Both HETP and OTP formed hydrogen bonds with the basic nitrogen of quinoline resin via their N—H functional groups. These polar electrostatic attractions resulted in an interaction energy of −26.3 kcal/mol for the OTP-resin interaction within the asphaltene-OTP-resin tertiary complex. The interaction energy for the HETP-resin interaction in the asphaltene-HETP-resin tertiary complex was −24.2 kcal/mol.

The binding strength of both OTP-asphaltene and HETP-asphaltene increased within the tertiary complexes. However, the HETP-asphaltene strength (−40.4 kcal/mol) remained lower than the asphaltene-asphaltene strength. In contrast, the OTP-asphaltene interaction in the tertiary system (−52.1 kcal/mol) reached a level of strength and stability comparable to one of the asphaltene-asphaltene complexes (−52.5 kcal/mol). This indicated that OTP-asphaltene and asphaltene-asphaltene interactions were competitive in the presence of resins.

The calculations suggested that HETP-asphaltene interactions do not reach a threshold capable of disrupting asphaltene-asphaltene binding or penetrating between asphaltenes to reduce or prevent agglomerations. This indicated that HETP was not likely to overcome the energy barrier to form a stable tertiary complex, such as the asphaltene-HETP-resin. The interaction of HETP appeared to target polar sites within the bitumen, rather than balancing the binder composition. This can lead to adverse effects, such as aggregation with polar molecules and uneven distribution of components within the binder mixture. In contrast, OTP disrupted a portion of the polar-polar attractions between asphaltenes, allowing its non-polar arms to penetrate between the asphaltenes. The hindrance caused at least in part by the non-polar arms of OTP and the side alkyl chains of asphaltene weakened the binding between asphaltenes.

The alkylation of asphaltene and the presence of additional non-polar alkyl chains can create repulsion between asphaltenes, preventing their aggregation. Therefore, the longer non-polar arms of OTP played a role in its distribution within the binder matrix.

Asphaltene-asphaltene interactions can possess a high interaction energy of −75.3 kcal/mol, suggesting that a portion of asphaltene in the binder likely exists in dimer forms. This suggested that OTP is capable of dissociating a portion of the asphaltene dimer plates with lower stability and preventing aggregation between asphaltene dimers or between asphaltene dimers and another asphaltene plate.

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

What is claimed is:

1. A method for depolymerizing polyester material to yield a terephthalamide, the method comprising:
combining the polyester material, an alkylamine, and a mediator to yield a reaction mixture, wherein the mediator comprises ethylene glycol;
heating the reaction mixture; and
maintaining a reaction for a reaction time to yield the terephthalamide.

2. The method of claim 1, wherein the polyester material comprises polyethylene terephthalate, polyethylene terephthalate-glycol, poly(trimethylene terephthalate), or any combination thereof.

3. The method of claim 1, wherein the alkylamine comprises butylamine, amylamine, hexylamine, heptylamine, octylamine, nonylamine, decylamine, undecylamine, dodecylamine, tridecylamine, tetradecylamine, pentadecylamine, hexadecylamine, heptadecylamine, octadecylamine, isobutylamine, isopentylamine, (2-methylbutyl)amine, 2-ethyl-1-hexylamine, or any combination thereof.

4. The method of claim 1, wherein the terephthalamide comprises N¹, N⁴-dioctylterephthalamide (OTP), N¹, N⁴-didodecylterephthalamide (DTP), N¹, N⁴-dihexadecylterephthalamide (HTP), or any combination thereof.

5. The method of claim 1, wherein a weight ratio of polyester:alkylamine:mediator in the reaction mixture is in a range of about 1:1:4 to 1:4:4.

6. The method of claim 1, wherein a molar ratio of polyester:alkylamine in the reaction mixture is in a range of 1:2 to 1:10.

7. The method of claim 1, wherein a molar ratio of polyester:mediator is in a range of 1:2 to 1:24.

8. The method of claim 1, wherein heating comprises heating to a temperature in a range of 100° C. to 200° C.

9. The method of claim 1, wherein heating comprises heating to a temperature in a range of 120° C. to 130° C.

10. The method of claim 1, wherein the reaction time is between 1 hour and 72 hours.

11. The method of claim 1, wherein the reaction time is between 1 hour and 10 hours.

12. The method of claim 1, wherein the reaction mixture is contained in a reaction vessel, and further comprising purging the reaction vessel with an inert gas.

13. The method of claim 12, wherein the inert gas comprises nitrogen.

14. The method of claim 1, further comprising isolating the terephthalamide.

15. The method of claim 14, wherein isolating the terephthalamide comprises combining the terephthalamide with a washing agent.

16. The method of claim 15, wherein the washing agent comprises acetone.

17. The method of claim 14, wherein isolating the terephthalamide comprises filtering or drying the terephthalamide.

18. The method of claim 1, wherein the combining, the heating, and the maintaining is performed continuously in an extruder.

19. A method of preparing a modified asphalt binder, the method comprising:
combining a terephthalamide produced by the method of claim 1 with an asphalt binder to yield a modified asphalt binder precursor; and
heating the modified asphalt binder precursor to yield a modified asphalt binder.

20. The method of claim 19, wherein a weight ratio of the asphalt binder to the terephthalamide is in a range of about 50:1 to about 10:1.
